(19) United States
(12) Patent Application Publication
(10) Pub. No.: US 2010/0279361 A1
(43) Pub. Date: Nov. 4, 2010

(54) TWO-STAGE METHOD FOR PRETREATMENT OF LIGNOCELLULOSIC BIOMASS

(75) Inventors: Colin R. South, Lexington, MA (US); Charles E. Wyman, Riverside, CA (US); Richard Lance Martin, San Francisco, CA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Mascoma Corporation, Lebanon, NH (US)

(21) Appl. No.: 12/598,169

(22) PCT Filed: May 2, 2008
(86) PCT No.: PCT/US08/62358

§ 371 Date: Jun. 30, 2010

Related U.S. Application Data
(60) Provisional application No. 60/915,503, filed on May 2, 2007.

(51) Int. Cl. C12P 19/04; C12P 7/10; C07C 31/08; C08B 1/00

(52) U.S. Cl. 435/101; 435/165; 568/840; 536/56

(57) ABSTRACT

One aspect of the invention relates to a process, comprising treating lignocellulosic biomass according to a first pretreatment protocol, thereby generating a first product mixture; separating the first product mixture into a first plurality of fractions; and treating at least one fraction of said first plurality of fractions according to a second pretreatment protocol, thereby generating a second product mixture. In one embodiment, the lignocellulosic biomass is selected from the group consisting of grass, switch grass, cord grass, rye grass, reed canary grass, miscanthus, sugar-processing residues, sugarcane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp protocol protocol fiber, paper sludge, sawdust, hardwood, softwood, and combinations thereof.

### RELATED APPLICATIONS

This application claims the benefit of priority to U.S. Provisional Patent Application Ser. No. 60/915,503, filed May 2, 2007.

### BACKGROUND OF THE INVENTION

Plant biomass is a natural resource for the biological conversion of energy to forms useful to humanity. Among forms of plant biomass, lignocellulosic biomass is particularly well-suited for energy applications because of its large-scale availability, low cost, and environmentally benign production. In particular, many energy production and utilization cycles based on lignocellulosic biomass have near-zero greenhouse gas emissions on a life-cycle basis.

Ethanol is the primary biologically-derived transportation fuel worldwide, with production mainly from corn in the U.S. and from sugarcane in Brazil. Domestic ethanol production currently decreases oil imports, reduces greenhouse gas emissions, and increases farm income, reducing federal crop support expenditures. The economics of corn ethanol production have been attractive over the last several years due to a combination of factors including low corn prices, high crude oil prices, technological improvements from over two decades of commercial production, government incentives, stable co-product prices, and demand stimulated by the renewable fuel standard passed as part of the energy policy act of 2005. With potential for two year investor payback periods on corn ethanol plants, the industry build-out has been bullish and production capacity has risen sharply from 3.6 billion gallons in 2004 to 5.1 billion gallons in the fall of 2006, with 3.6 billion gallons of additional capacity under construction. In 2006, ethanol production consumed 20% of the U.S. corn crop, and accounted for about 2% of U.S. fuel consumption for light-duty vehicles.

The rapid growth of the industry, however, has increased demand for corn, and as a result corn prices have risen from an average of $2.30 per bushel over the last 5 years, and $1.95 per bushel in 2006, to over $3.50 per bushel in the spring of 2007. While high corn prices are advantageous for corn growers, they reduce the profitability of ethanol production as well as other agricultural activities that consume corn, such as pork, animal feed, and poultry production. Moreover, environmental advocacy organizations, such as the NRDC and World Wildlife Fund, are concerned about the water quality and soil fertility implications of increased corn planting.

Independent of the status and future prospects of the corn ethanol industry, ethanol production from cellulosic biomass, such as wood, grass, and agricultural residues, has attracted a great deal of attention of late. Although cellulosic ethanol is not yet produced commercially, projected features include a decisively positive fossil fuel displacement ratio, near-zero net greenhouse gas emissions, potential for substantial soil fertility and carbon sequestration benefits, and feedstocks with broad geographical diversity, expected to be widely available at a cost per unit energy (e.g. $/GJ) equal to that provided by oil were it available at about $17/barrel. Several studies foresee the possibility of cellulosic ethanol playing a large role in meeting national mobility demands, particularly when combined with improved vehicle efficiency. Unprecedented investments in support of cellulosic biomass have recently been made by both the government and the private sector.

Efforts to produce ethanol by biological and thermo-chemical processes are receiving increased attention. Thermo-chemical processes use heat, pressure, and steam to convert feedstock into synthesis gas (“syngas”). Syngas is passed over a catalyst and transformed into alcohols such as ethanol. Biological processes to convert cellulosic biomass into ethanol involve pretreatment, production of reactive carbohydrate, and biological conversion, in which the carbohydrate is converted into ethanol. The beer output from biological conversion contains ethanol and non-fermented solids, which are both recovered for storage and sale in downstream processing.

In the corn ethanol space, the ICM process is generally considered to be the industry standard due to the number of operating dry mills using the company's design. In stark contrast, there is no standard practice in the emergent and immature cellulosic ethanol space. Industry leaders are exploring different process configurations designed around different cellulosic feedstocks. In other words, the choice of cellulosic feedstock tends to drive design. All cellulosic feedstocks have similar components, but vary in composition and bulk density. These differences will impact the design and configuration of equipment required to produce reactive carbohydrate.

Independent of feedstock-specific design, however, production of reactive carbohydrate will necessarily involve a pre-treatment process with a catalyst, such as acid or steam, to improve the enzymatic digestibility of the five-carbon (hemi-cellulose) and six carbon (cellulose) structural sugars in the naturally recalcitrant cellulosic material; the recalcitrance results from the crystalline architecture of cellulose fibrils, which are sheathed in lignin and hemicellulose. Pretreatment exposure time, temperature, and pH are the variables that determine the extent to which the cellulosic carbohydrate fractions are cleaved and thereby rendered amenable to enzymatic hydrolysis in subsequent biological conversion steps.

Some cellulosic processes pretreat at higher temperatures, for longer residence times, and at lower pH (so-called “severe conditions”) to initiate a greater extent of hydrolysis, which typically reduces the additional enzyme loading required in subsequent steps to liberate soluble monomers that can be fermented. Often, acid is used as a catalyst in these pretreatment processes, which have proven effective in achieving high total sugar yields. For example, favorable results have been obtained from a pretreatment protocol with dilute aqueous sulfuric acid (about 1.0 to about 2.0% acid); temperatures of about 160° C. to about 200° C.; and times from about 5 to about 20 minutes. Under these conditions, about 80-90% of the hemicellulose sugars can be recovered from pretreatment, and enzymes can digest the cellulose in the residual solids to glucose with high yields (about 90%).

“Severe” pre-treatment conditions, however, have several drawbacks. First, in this severity range the hemicellulose sugars form degradation products, which reduce the efficiency of eventual fermentation. Second, the high temperatures, high pressures, and acidic conditions require expensive reaction vessels to avoid corrosion. Third, the high pressures present difficulties in continuously feeding the solids to the pre-treatment device. An additional drawback is the generation of acidic waste. Accordingly, use of a severe pretreatment protocol may necessitate costly adjustment of the pH of or removal of certain byproducts from the pretreated material prior to biological fermentation to ethanol.

“Mild” pretreatment protocols rely on less severe conditions, typically with an eye towards reducing the equipment costs. However, the product stream from mild pre-treatment typically includes a greater proportion of carbohydrate oligomers, which creates a downstream requirement for higher enzyme loading to liberate soluble monomers prior to biological conversion to ethanol. Steam has been shown to be effective for pretreatment of cellulose materials that contain acetylated pentosans, such as xylan. The steam hydrolyzes the acetyl groups, resulting in acetic acid, which initiates hydrolysis of hemi-cellulose polymers. This “auto-hydrolysis” process may be operated at a range of conditions, including but not limited to 210° C. with 5 to 20 minute of residence time. High cellulose (e.g., glucan) recovery is achieved with auto-hydrolysis under these conditions, which means that much of the cellulose is either hydrolyzed to monomers in pre-treatment or amenable to enzymatic hydrolysis and fermentation. However, in this severity range the hemicellulose sugars form degradation products, which reduce the efficiency of fermentation. More importantly, it is challenging to continuously feed solids to a reactor operating at the high pressure corresponding to a saturation temperature of 210° C. At lower temperatures, such as 190° C., high hemicellulose (xylan) recovery results with reduced degradation. However, only a small fraction of cellulose can be recovered, thereby limiting the overall process yield in terms of gallons of ethanol produced per mass unit feedstock.

Other products like furfural, levulinic acid, and lignin can also be produced from lignocellulose. As described above, lignocellulose splits into lignin and a cellulosic component when subjected to acid treatment. The cellulosic component can hydrolyze to its constituent pentose and hexose monomers. The pentose monomers, upon further acid treatment, can degrade to furfural, and the hexose monomer can degrade to hydroxymethylfurfural. Hydroxymethylfurfural can degrade still further in the presence of acid to levulinic acid. Furfural is used primarily in lubricating oil manufacture and in making resins. Levulinic acid is also used to make resins, and, in addition, plasticizers, fragrance products, and pharmaceuticals. Lignin is used in making vanillin and as a filler and binder in some resin products.

### SUMMARY OF THE INVENTION

Aspects of the present invention relate to the recovery of hemicellulose and cellulose carbohydrate fractions in a sequence that keeps materials of construction to a minimum by addition of no external chemicals; minimizing the presence of inhibitory degradation products which maximizes fermentation efficiency; and bypassing the technical challenges associated with feeding lignocellulosic materials to a high-pressure pretreatment device.

One aspect of the present invention relates to pre-treatment and fermentation modules, each operating at conditions optimized for recovery of the two primary carbohydrate fractions, enabling high recovery of both without formation of degradation products.

In certain embodiments, a first module operates at low severity, enabling continuous solids feeding to mild pressure using proven equipment, followed by a second module operating at high severity fed by a pump designed for high solids slurry.

In certain embodiments, the carbohydrate fractions of cellulosic biomass are recovered in a step-wise manner in two operating modules such that the first module targets the fraction recovered at low severity, namely the hemi-cellulose. In certain embodiments, degradation products are minimized because the first module operates at mild conditions. The recovered sugars are hydrolyzed with enzyme, fermented, and the ethanol is stripped from the solids. Since enzymatic hydrolysis and fermentation reduce the viscosity, the slurry is pumped to the second module, thus bypassing the concern associated with feeding solids to high pressure. In certain embodiments, pumping eases the operability of the second module, which targets the carbohydrate fraction recovered at higher severity, namely the cellulose. In certain embodiments, degradation products do not form because the hemicellulose has already been recovered and the sugars fermented. Recovered cellulose sugars are hydrolyzed with enzyme, fermented, and the ethanol is stripped from the solids.

In certain embodiments, aspects of the present invention are related to the pretreatment of lignocellulosic biomass in a two-stage process to recover sugars from hemicellulose and cellulose with high yields. In certain embodiments, the sugars provide valuable building blocks for biological conversion or chemical conversion to a wide range of products. In certain embodiments, the products include ethanol. In certain embodiments, the products include levulinic acid. In certain embodiments, the products include furfural. In certain embodiments, the products include lignin.

In certain embodiments, the two-stage pretreatment methodology of present invention mitigates some of the problems associated with deleterious degradation due to the presence of fermentation inhibitors.

### BRIEF DESCRIPTION OF THE FIGURES

FIG. 1 depicts a table reporting selected protocols used for the pretreatment of lignocellulosics.

FIG. 2 depicts schematically a general two-stage methodology for the pretreatment of lignocellulosic biomass materials.

FIG. 3 depicts schematically a modular ethanol production plant (MOD-1).

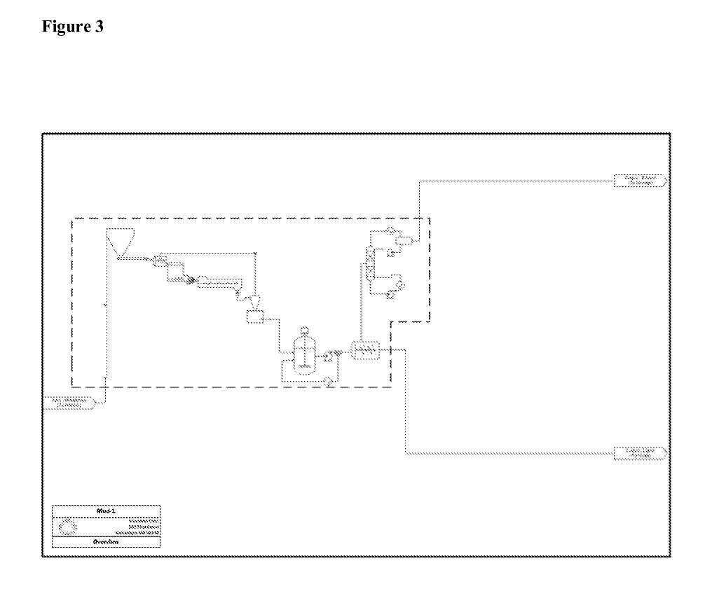

FIG. 4 depicts schematically the clip on to a modular ethanol production plant (MOD-2).

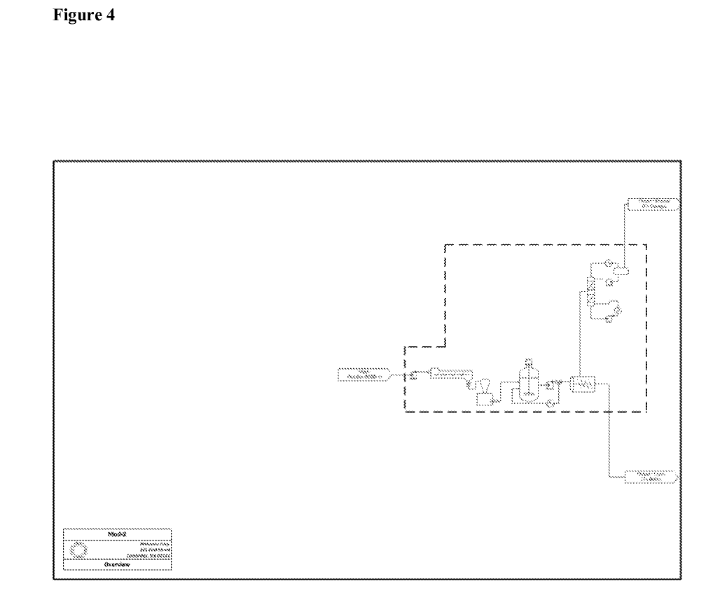

FIG. 5 depicts schematically the integration of MOD-1 and MOD-2.

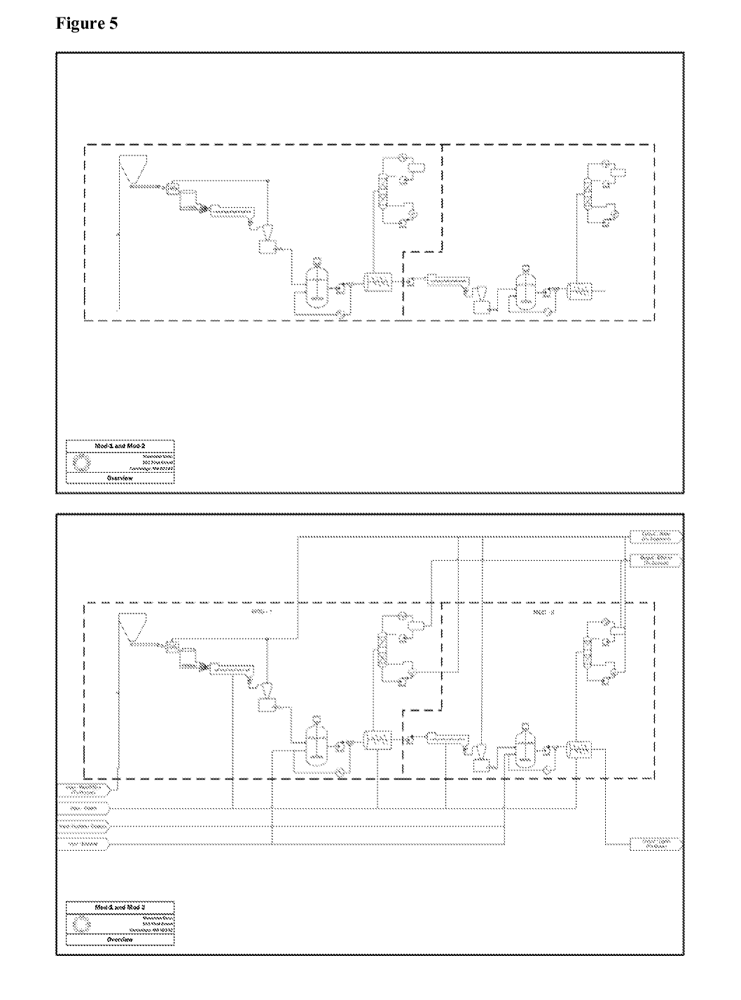

FIG. 6 depicts schematically a general biologically-based process configuration for production of ethanol and other products from lignocellulosic biomass.

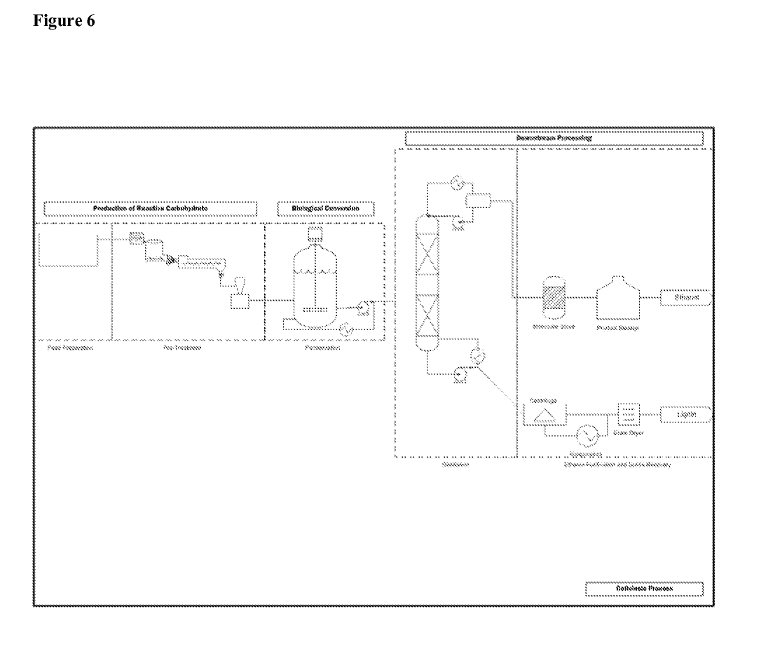

### DETAILED DESCRIPTION OF THE INVENTION

Aspects of the present invention relate to a process by which the cost of producing ethanol or other fine chemicals from cellulosic biomass-containing materials can be reduced by using a novel processing configuration. In certain embodiments, the present invention relates to a two-stage pretreatment process, wherein a first-stage separation of cellulose materials from other biomass components (e.g., hemicellulose), target products (e.g., ethanol), and deleterious side products (e.g., fermentation inhibitors) mitigates the problems associated with deleterious degradation and downstream loss of yield.

The incorporation of a two-stage method of pretreatment in the processing of lignocellulosic biomass raw materials improves process economics without sacrificing yield of a target product. The recovery of sugars from hemicellulose and cellulose with high yields provides valuable building blocks for biological conversion or chemical conversion to a wide range of products, including ethanol for use as a transportation fuel and levulinic acid.

### Definitions

As used herein, the term “biomass” refers to a primarily carbohydrate-containing material. Biomass can also refer to a polysaccharide-containing material. It can also refer to a cellulose-, hemicellulose-, or lignocellulose-containing material. Biomass is commonly obtained from, for example, wood, plants, residue from agriculture or forestry, organic component of municipal and industrial wastes, primary sludges from paper manufacture, waste paper, waste wood (e.g., sawdust), agricultural residues such as corn husks, corn cobs, rice hulls, straw, bagasse, starch from corn, wheat oats, and barley, waste plant material from hard wood or beech bark, fiberboard industry waste water, bagasse pity, bagasse, molasses, post-fermentation liquor, furfural still residues, aqueous oak wood extracts, rice hull, oats residues, wood sugar slops, fir sawdust, naphtha, corncob furfural residue, cotton balls, rice, straw, soybean skin, soybean oil residue, corn husks, cotton stems, cottonseed hulls, starch, potatoes, sweet potatoes, lactose, waste wood pulping residues, sunflower seed husks, hexose sugars, pentose sugars, sucrose from sugar cane and sugar beets, corn syrup, hemp, and combinations of the above.

The terms “lignocellulosic material,” “lignocellulosic substrate,” and “lignocellulosics” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants, sugar-processing residues, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; and forestry wastes, such as but not limited to recycled wood pulp fiber, sawdust, hardwood (e.g., poplar, oak, maple, birch), softwood, or any combination thereof

“Lignocellulosic material” may comprise one species of fiber; alternatively, lignocellulosic material may comprise a mixture of fibers that originate from different lignocellulosic materials. Particularly advantageous lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

“Paper sludge” is also a viable feedstock for ethanol production. Paper sludge is solid residue arising from pulping and paper-making, and is typically removed from process wastewater in a primary clarifier. At a disposal cost of $30/wet ton, the cost of sludge disposal equates to $5/ton of paper that is produced for sale. The costly alternative of disposing wet sludge at this price is a significant incentive to convert the material for other uses, such as conversion to ethanol.

The terms “reactor” and “pretreatment reactor” used herein mean any vessel suitable for practicing a method of the present invention. The dimensions of the pretreatment reactor should be sufficient to accommodate the lignocellulose material conveyed into and out of the reactor, as well as additional headspace around the material. In a non-limiting example, the headspace extends about one foot around the space occupied by the materials. Furthermore, the pretreatment reactor should be constructed of a material capable of withstanding the pretreatment conditions. Specifically, the construction of the reactor should be such that the pH, temperature and pressure do not affect the integrity of the vessel.

### Pretreatment Protocols

Lingocellulosic materials require pretreatment to increase the accessibility of hemicellulose, cellulose, and other components for further processing. In certain embodiments, further processing includes enzymatic hydrolysis.

The native structure of lignocellulosics inhibits degradation. In addition to cellulose's highly-resistant crystalline structure, the lignin surrounding the cellulose forms a physical barrier. Accordingly, the sites available for attack (e.g., by enzymes) are limited. One idealized outcome of pretreatment, therefore, would be to reduce lignin content with a concomitant reduction in crystallinity and increase in surface area.

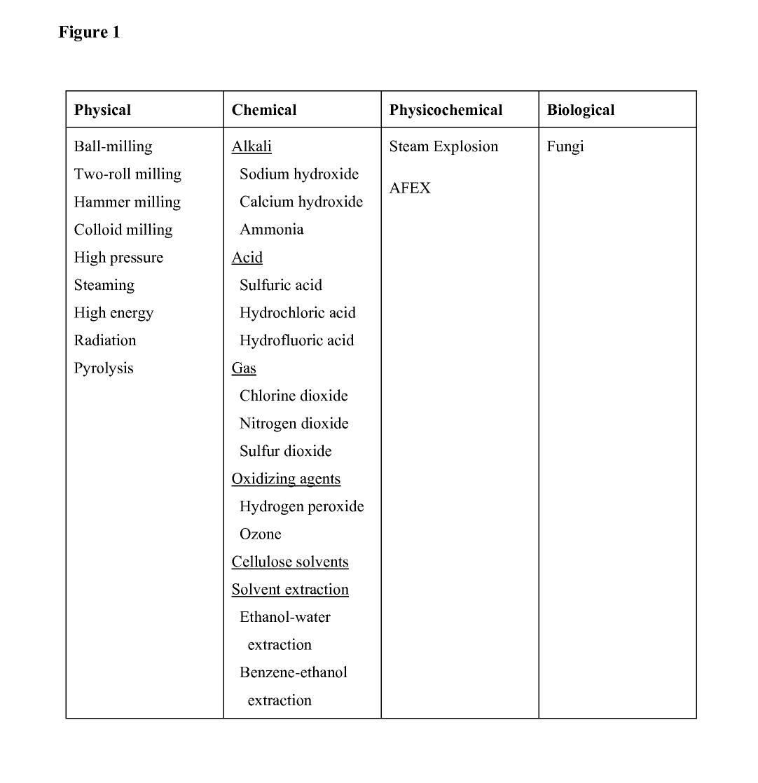

Pretreatment protocols can be classified as physical, chemical, physicochemical, or biological. A selected sample of various pretreatment protocols that have been used to increase lignocellulosic digestibility are summarized in FIG. 1. A further discussion of these pretreatments can be found in Holtzapple et al. (U.S. Pat. No. 5,865,898, which is hereby incorporated by reference). In certain embodiments, aspects of the present invention relate to the application of such pretreatment protocols within the construct of a two-stage pretreatment methodology.

Among processes developed to pretreat lignocellulosic biomass, as noted in FIG. 1, steam-explosion has been identified as a low cost and high yield technology, along with low-pressure steam autohydrolysis. Steam explosion heats wetted lignocellulose to high temperatures (e.g., about 160° C. to about 230° C.) and releases the pressure immediately. Rapid decompression flashes the water trapped in the fibers, which leads to a physical size reduction. The elevated temperatures remove acetic acid from hemicellulose which allows some autohydrolysis of the biomass. In certain embodiments, additional chemical agents, such as sulfuric acid or ammonia (e.g., gaseous, anhydrous liquid, or ammonium hydroxide), may be added to aid in the hydrolysis. In certain embodiments, the pretreated cellulose can then be sterilized to prevent growth of other microorganisms during the fermentation reaction.

Another physicochemical pretreatment is Ammonia Fiber Explosion (AFEX). AFEX requires soaking the lignocellulose in liquid ammonia at high pressure, followed by an explosive release of the pressure. Pretreatment conditions (about 30° C. to about 100° C.) are less severe than steam explosion. An increase in accessible surface area coupled with reduced cellulose crystallinity (caused by ammonia contacting) result in increased enzymatic digestibility.

For example, the use of ammonia under pressure to increase protein availability and cellulosic digestibility of a cellulosic containing plant material (alfalfa) is described in Hultquist (U.S. Pat. No. 4,356,196; hereby incorporated by reference). Liquid ammonia impregnates the plant material, which is explosively released upon being exposed upon rapid pressure release. The resulting processed material is used for ethanol production or as a feedstock for food or dairy animals.

In addition, Dale et al. (U.S. Pat. Nos. 4,600,590 and 5,037,663; each incorporated by reference) describes the use of various volatile chemical agents, particularly ammonia, to treat the cellulose containing materials. Further, Holzapple et al. (U.S. Pat. No. 5,171,592; which is incorporated by reference) describes an AFEX process wherein the treated biomass product is stripped of residual swelling agent with super-heated vapors.

AFEX processes are also described in European Patent No. 0 077 287; Dale, B. E., et al., Biotech. and Bioengineering Symp. No. 12, 31-43 (1982); Dale, B. E., et al., Developments in Industrial Microbiology, 26 (1985); Holtzapple, M. T., et al. Applied Biochem. and Biotech. 1991, 28/29, 59-74; Blasig, J. D., et al. Resources, Conservation and Recycling 1992, 7, 95-114; Reshamwala, S., et al. Applied Biochem. and Biotech. 1995, 51/52, 43-55; Dale, B. E., et al. Bioresource Tech. 1996, 56, 111-116; and Moniruzzaman, M., et al. Applied Biochem. and Biotech. 1997, 67, 113-126; all of which are incorporated by reference. Additional examples can found in the references cited in Holtzapple et al. (U.S. Pat. No. 5,865,898; hereby incorporated by reference).

Pretreatment of biomass using ammonia impregnation typically involves a number of steps. Vaporized ammonia may be recycled in a low pressure vessel. Sulfur dioxide-catalyzed steam explosion processes may also be employed using a multi-step protocol. The sulfur dioxide may also be recycled.

In certain embodiments, the lignocellulosic materials may be soaked in water or other suitable liquid(s) prior to the addition of steam or ammonia or both, or steam or sulfur dioxide or both. In certain embodiments, the excess water may be drained off the lignocellulosic materials. In certain embodiments, the soaking may be done prior to conveying into a reactor, or subsequent to entry (i.e., inside a pretreatment reactor).

The size range of the substrate material varies widely and depends upon the type of substrate material used as well as the requirements and needs of a given process. In certain embodiments, the lignocellulosic raw material may be prepared in such a way as to permit ease of handling with conveyors, hoppers and the like. In the case of wood, the chips obtained from commercial chippers are suitable; in the case of straw it is sometimes desirable to chop the stalks into uniform pieces about 1 to about 3 inches in length. In certain embodiments, depending on the intended degree of pretreatment, the size of the substrate particles prior to pretreatment may range from less than a millimeter to inches in length.

In certain embodiments, ultrasound treatments may be applied to processes of the present invention. See U.S. Pat. No. 6,333,181, which is hereby incorporated by reference.

### Two-Stage Pretreatment Methodology

Aspects of the present invention relate to the development of a two-stage approach to the pretreatment of cellulosic biomass. In certain embodiments, an objective of this process is to maximize recovery of sugars at low pressures and subsequently augment the digestibility of cellulose in pretreated solids. In certain embodiments, lignocellulosic biomass materials can be pretreated by any number of protocols; for example, suitable pretreatment protocols include but are not limited to those described above and tabulated in FIG. 1.

In certain embodiments, in a first stage processing a significant portion of the composite hemicellulose is released as sugars. In certain embodiments, the pretreated mixture is transferred and/or otherwise separated to afford a fraction containing much of the hemicellulose-derived sugars and/or residual chemicals from pretreatment.

In certain embodiments, in a second stage processing the remaining fraction, comprising mostly cellulose, is further treated to enhance the enzymatic digestibility of the remaining cellulose. In certain embodiments, operation of these mild, moderate conditions reduces the pressure and construction costs while also avoiding problems associated with the feeding of solids in currently practiced processing protocols. In certain embodiments, separating the so-called cellulose fraction from deleterious side products (e.g., fermentation inhibitors) mitigates the problems associated with undesirable degradation and downstream loss of yield.

In certain embodiments, the results of either of the two stages in this pretreatment method can be further processed and/or prepared appropriately in downstream treatments and/or alternative protocols. In certain embodiments, for example, solids from the second stage are subsequently be subjected to enzymatic hydrolysis to release most of the remaining sugars. In certain embodiments, the fraction from the first stage is conditioned as necessary for the sugars to be biologically or chemically converted to a variety of products. In certain embodiments, a product is ethanol produced by fermentation.

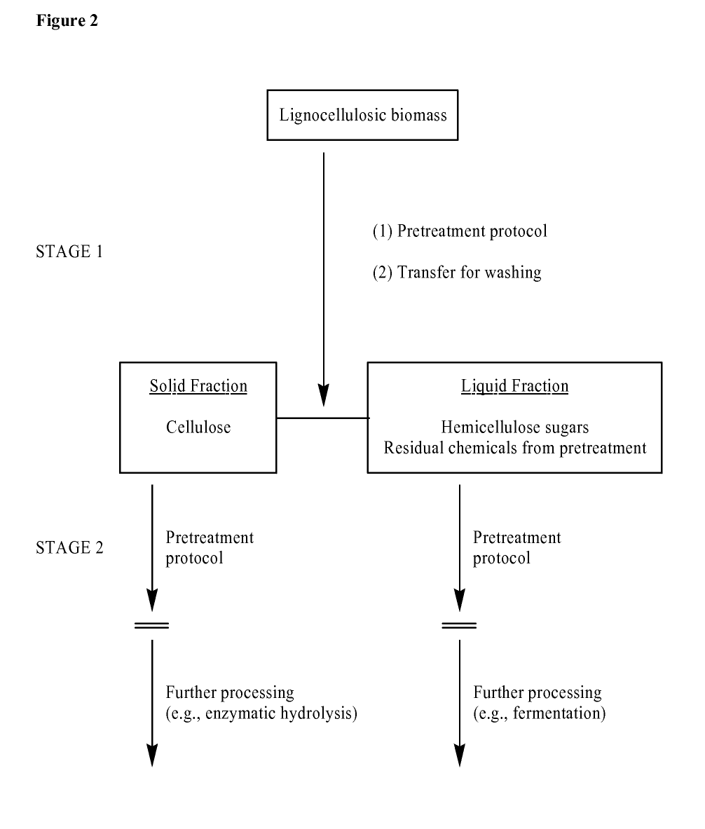

FIG. 2 depicts generally a representative and non-limiting schematic of such a two-stage process. In certain embodiments, lignocellulosic biomass materials such as corn stover, sugarcane bagasse, switchgrass, and poplar wood are heated to about 100 to about 140° C. in a solution of about 2 to about 5% sulfuric acid and held for a sufficient time (about 30 to about 90 minutes) to release most of the hemicellulose into solution. In certain embodiments, the pretreated mixture is transferred to washing equipment to remove the liquid fraction containing much of the hemicellulose sugars and the acid from the solids. In certain embodiments, the separation occurs near the prior reaction temperature to reduce heat input demands. In certain embodiments, the solids are added to a second reactor and heated to a higher temperature of about 160° C. to about 220° C. to enhance the digestibility of the remaining cellulose by enzymes. In certain embodiments, this addition is done with acid. The solids are cooled, prepared appropriately, and transferred to an enzymatic hydrolysis step for release of most of the remaining sugars.

In certain embodiments, the separation of the hemicellulose, hemicellulase, and other residual pretreatment chemicals, such as acid in the first pretreatment stage, via liquification provides an number of advantages. The exemplary stage 1 pretreatment protocol utilizing a sulfuric acid solution may lead to the production of acidic wastes, and the formation of toxic compounds that can hinder subsequent microbial fermentations. For example, several degradation products, such as furfural, hydroxymethylfurfural (HMF), phenols, and formic, acetic and other acids produced during the pretreatment and hydrolysis can inhibit the fermentation of the remaining cellulose ‘solid fraction’, eventually affecting yields. In certain embodiments, the first stage separation effectively removes and/or otherwise mitigates these problems associated with deleterious degradation from fermentation inhibitors without compromising yield

In certain embodiments, the “liquid fraction” is subjected to a further second stage pretreatment protocol. In certain embodiments, this protocol involves dilute acid hydrolysis. In certain embodiments, the sugars are conditioned to be biologically or chemically converted to a variety of products, such as fine chemicals, and including ethanol by fermentation. In certain embodiments, the “solid fraction” is subjected to a further second stage pretreatment protocol to enhance the enzymatic digestibility of the remaining cellulose. In certain embodiments, following this two-stage pretreatment process, said ‘solids’ can be further processed by known methods. In certain embodiments, the method is enzymatic hydrolysis.

### Additional Process Strategies

In certain embodiments, aspects of the present invention may be applicable with the process known as consolidated bioprocessing (CBP). CBP is a processing strategy for cellulosic biomass that involves consolidating into a single process step four biologically-mediated events: enzyme production, hydrolysis, hexose fermentation, and pentose fermentation. Implementing this strategy requires the development of microorganisms that both utilize cellulose, hemicellulose, and other biomass components while also producing a product of interest (e.g., ethanol) at sufficiently high yield and concentrations. The feasibility of CBP is supported by kinetic and bioenergetic analysis. See van Walsum and Lynd Biotech. Bioeng. 1998, 58, 316.

An approach to organism development for CBP involves conferring the ability to grow on lignocellulosic materials upon microorganisms that naturally have high product yield and tolerance via expression of a heterologous cellulasic system and perhaps other features. For example, Saccharomyces cerevisiae has been engineered to express over two dozen different saccharolytic enzymes. See Lynd et al. Microbiol. Mol. Biol. Rev. 2002, 66, 506. Such recombinant microorganisms have the ability to produce cellulase and/or hemicellulase enzymes to hydrolyze more specifically the cellulose and/or hemicellulose portions of lignocellulosic biomass materials, respectively. For example, hemicelluloses are heteropolysaccharides formed from a variety of monomers. The most common monomers are glucose, galactose, and mannose (hexoses) and xylose and arabinose (pentoses). Hemicellulase enzymes are categorized (e.g., as a glucanase, xylanase, or mannanase) based on their ability to catalyze the hydrolysis of heteropolysaccharides composed of glucan, xylan, or mannan, respectively.

In certain embodiments, aspects of the present invention may be applicable with the process known as simultaneous saccharification and fermentation (SSF), which is intended to include the use of said microorganisms and/or one or more recombinant hosts (or extracts thereof, including purified or unpurified extracts) for the contemporaneous degradation or depolymerization of a complex sugar (i.e., cellulosic biomass) and bioconversion of that sugar residue into ethanol by fermentation.

### Ethanol Production

Since pentose sugars are abundant, the fermentation of xylose and other hemicellulose constituents is an attractive option for the development of an economically viable process to produce ethanol from biomass. Hexose (C6) and pentose (C5) sugars are converted into pyruvate by modified glycolytic pathways. The pyruvate can then be converted to ethanol. For example, the net reaction for a pentose sugar is typically such that three pentose sugars yield five molecules of ethanol and five molecules of carbon dioxide. Aspects of the present invention relate to the use of ethanologenic enzymes (i.e., pyruvate decarboxylase and/or alcohol dehydrogenase).

A variety of microorganisms are known to be useful for the conversion of organic material to ethanol. Examples of microorganisms which may be used in practice are fermentation agents, such as Saccharomyces cerevisiae for producing ethanol. An alternative ethanol-producing organism which may be used is Zymomonas mobilis or a member selected from the Zymomonas, Erwinia, Klebsiella, Xanthomonas or Escherichia genii. Other microorganisms that convert sugars to ethanol include species of Schizosaccharomyces (such as S. pombe), Pichia (P. stipitis), Candida (C. shehatae) and Pachysolen (P. tannophilus).

For the production of ethanol, microorganisms can also be engineered with nucleic acids, such as those disclosed in U.S. Pat. No. 5,000,000, which is hereby incorporated by reference. A biocatalyst, such as a recombinant ethanologenic bacterium, can be engineered to express one or more enzymatic activities, such as those described above, in particular amounts sufficient for degrading complex sugars. Such a biocatalyst would be suitable for the efficient degradation of complex sugars and subsequent fermentation into alcohol.

In certain embodiments, transformed or recombinant Gram-positive bacteria, which encode microbes with the ability to produce ethanol as a fermentation product, are also applicable in the downstream processes. See U.S. Pat. Nos. 5,916,787 and 5,482,846, which are hereby incorporated by reference. In certain embodiments, for example, a Gram-positive bacterial host, such as Bacillus subtillis or Bacillus polymyxa, can be transformed with (1) heterologous Zymomonas mobilis genes encoding alcohol dehydrogenase and pyruvate decarboxylase, wherein said genes are expressed at sufficient levels to result in the production of ethanol as a fermentation product; and (2) a heterologous DNA segment encoding a protein involved in transcription of mono- and oligosaccharides into the host cell. One skilled in the art can readily identify a variety of additional suitable microbial systems which may be used.

The U.S. Department of Energy (DOE) provides a method for calculating theoretical ethanol yield. Accordingly, if the weight percentages are known of C6 sugars (i.e., glucan, galactan, mannan), the theoretical yield of ethanol in gallons per dry ton of total C6 polymers can be determined by applying a conversion factor as follows:

(1.11 pounds of C6 sugar/pound of polymeric sugar)×(0.51 pounds of ethanol/pound of sugar)×(2000 pounds of ethanol/ton of C6 polymeric sugar)×(1 gallon of ethanol/6.55 pounds of ethanol)×(1/100%), wherein the factor (1 gallon of ethanol/6.55 pounds of ethanol) is taken as the specific gravity of ethanol at 20° C.

And if the weight percentages are known of C5 sugars (i.e., xylan, arabinan), the theoretical yield of ethanol in gallons per dry ton of total C5 polymers can be determined by applying a conversion factor as follows:

(1.136 pounds of C5 sugar/pound of C5 polymeric sugar)×(0.51 pounds of ethanol/pound of sugar)×(2000 pounds of ethanol/ton of C5 polymeric sugar)×(1 gallon of ethanol/6.55 pounds of ethanol)×(1/100%), wherein the factor (1 gallon of ethanol/6.55 pounds of ethanol) is taken as the specific gravity of ethanol at 20° C.

It follows that by adding the theoretical yield of ethanol in gallons per dry ton of the total C6 polymers to the theoretical yield of ethanol in gallons per dry ton of the total C5 polymers gives the total theoretical yield of ethanol in gallons per dry ton of feedstock.

Applying this analysis, the DOE provides the following examples of theoretical yield of ethanol in gallons per dry ton of feedstock: corn grain, 124.4; corn stover, 113.0;

rice straw, 109.9; cotton gin trash, 56.8; forest thinnings, 81.5; harwood sawdust, 100.8; bagasse, 111.5; and mixed paper, 116.2. It is important to note that these are theoretical yields. The DOE warns that depending on the nature of the feedstock and the process employed, actual yield could be anywhere from 60% to 90% of theoretical, and further states that “achieving high yield may be costly, however, so lower yield processes may often be more cost effective.”

Remarkably, aspects of the present invention relate to improvements in process economics without sacrificing foreseeable ethanol yield. Because cheaper construction materials may be used, pretreatment capital costs are reduced considerably if severe conditions are not required. This approach does not reduce the ethanol yield because it achieves the same the results associated with acidic and/or high temperature pretreatment. It is recognized that without aggressive pretreatment conditions, fractional separation of the biomass may not be complete.

Processes provided by the present invention are widely applicable. Moreover, saccharification and/or fermentation products generated utilizing the two-stage pretreatment methods may be used to produce not only ethanol, but also higher value added chemicals, such as organic acids, aromatics, esters, acetone and polymer intermediates. For example, downstream processing may be targeted to furnish levulinic acid, a so-called platform chemical, which may be converted to a variety of other chemicals, including direct substitutions for petrochemicals, such as methyl tetrahydrofuran (MTHF), an oxygenated fuel additive that is becoming increasingly important. The U.S. Department of Energy has approved MTHF as a component in “P-series” alternative fuels, for which a large market exists. Use of the MTHF derived from levulinic acid greatly reduces waste and net energy consumption.

When lignocellulosic materials are degraded to constituent pentose and hexose monomers, the pentose monomers, upon further acid treatment, can degrade to furfural, and the hexose monomers can degrade to hydroxymethylfurfural. Hydroxymethylfurfural can degrade still further in the presence of acid to afford levulinic acid and formic acid. A method for the production of levulinic acid from the furfural by-product of lignocellulose degradation is presented in U.S. Pat. No. 4,897,947, which is hereby incorporated by reference. U.S. Pat. No. 4,236,021, which is hereby incorporated by reference, discloses a method of preparing levulinic acid from furfuryl alcohol. U.S. Pat. No. 3,663,368, which is hereby incorporated by reference, discloses a method of removing levulinic acid with microorganisms. U.S. Pat. No. 5,859,263, which is hereby incorporated by reference, describes a process for producing levulinic acid by extrusion of mixture of starch, water and mineral acid in a screw extruder. U.S. Pat. No. 5,608,105, which is hereby incorporated by reference, describes a process for producing levulinic acid by hydrolyzing a dilute concentration of carbohydrate-containing material in a mineral acid at high temperatures. U.S. Pat. Nos. 7,153,996 and 6,054,611, which are hereby incorporated by reference, describe production of levulinic acid from sugars produced as a result of acid hydrolysis.

### Cattle Feed

In addition to chemical production, lignocellulose can also be used as inexpensive cattle feed. Since raw lignocellulose cannot be easily digested by cattle, it must be processed to improve its digestibility before it can be fed to ruminants. Also, anaerobic fermentation using rumen microorganisms can produce low molecular weight volatile fatty acids.

### Exemplary Embodiments

In certain embodiments, the invention relates to a process, comprising:
(a) treating lignocellulosic biomass according to a first pretreatment protocol, thereby generating a first product mixture;
(b) separating the first product mixture into a first plurality of fractions; and
(c) treating at least one fraction of said first plurality of fractions according to a second pretreatment protocol, thereby generating a second product mixture.

In certain embodiments, the invention relates to the aforementioned process, wherein said lignocellulosic biomass is selected from the group consisting of corn stover, sugarcane bagasse, switchgrass, and poplar wood.

In certain embodiments, the invention relates to the aforementioned process, wherein said lignocellulosic biomass is corn stover.

In certain embodiments, the invention relates to the aforementioned process, wherein said lignocellulosic biomass is sugarcane bagasse.

In certain embodiments, the invention relates to the aforementioned process, wherein said lignocellulosic biomass is switchgrass.

In certain embodiments, the invention relates to the aforementioned process, wherein said lignocellulosic biomass is poplar wood.

In certain embodiments, the invention relates to a process, comprising:
(a) treating lignocellulosic biomass according to a first pretreatment protocol, thereby generating a first product mixture;
(b) separating the first product mixture into a first plurality of fractions; and
(c) treating at least one fraction of said first plurality of fractions according to a second pretreatment protocol, thereby generating a second product mixture; wherein
said lignocellulosic biomass is selected from the group consisting of grass, switch grass, cord grass, rye grass, reed canary grass, miscanthus, sugar-processing residues, sugarcane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, and combinations thereof.

In certain embodiments, the invention relates to the aforementioned process, wherein said first pretreatment protocol or second pretreatment protocol comprises ball-milling, two-roll milling, hammer milling, colloid milling, high pressure, steaming, high energy, radiation, pyrolysis, sodium hydroxide, calcium hydroxide, ammonia, sulfuric acid, hydrochloric acid, hydrofluoric acid, chlorine dioxide, nitrogen dioxide, sulfur dioxide, hydrogen peroxide, ozone, cellulose solvents, ethanol-water extraction, benzene-ethanol extraction, steam explosion, AFEX, recombinant microorganisms, or a combination thereof.

In certain embodiments, the invention relates to the aforementioned process, wherein said first pretreatment protocol comprises heating the lignocellulosic materials to a temperature in a solution of acid for a period of time.

In certain embodiments, the invention relates to the aforementioned process, wherein

said first pretreatment protocol comprises heating the lignocellulosic materials to a temperature in a solution of acid for a period of time; and said acid is sulfuric acid.

In certain embodiments, the invention relates to the aforementioned process, wherein said first pretreatment protocol comprises heating the lignocellulosic materials to a temperature in a solution of acid for a period of time; and said temperature is about 100° C. to about 140° C.

In certain embodiments, the invention relates to the aforementioned process, wherein said first pretreatment protocol comprises heating the lignocellulosic materials to a temperature in a solution of acid for a period of time; and said period of time is about 30 minutes to about 90 minutes.

In certain embodiments, the invention relates to the aforementioned process, wherein said first pretreatment protocol comprises heating the lignocellulosic materials to a temperature in a solution of acid for a period of time; and said solution of acid is of a concentration of about 2% to about 5%.

In certain embodiments, the invention relates to the aforementioned process, wherein said first pretreatment protocol comprises heating the lignocellulosic materials to a temperature in a solution of acid for a period of time; said solution of acid is of a concentration of about 2% to about 5%; and said acid is sulfuric acid.

In certain embodiments, the invention relates to the aforementioned process, wherein said separation into a first plurality of fractions comprises washing to remove a liquids fraction, thereby leaving a solids fraction.

In certain embodiments, the invention relates to the aforementioned process, wherein said separation into a first plurality of fractions comprises washing to remove a liquids fraction, thereby leaving a solids fraction; and said solids fraction comprises cellulosic materials.

In certain embodiments, the invention relates to the aforementioned process, wherein said separation into a first plurality of fractions comprises washing to remove a liquids fraction, thereby leaving a solids fraction; and said liquids fraction comprises mainly hemicellulosic materials in solution.

In certain embodiments, the invention relates to the aforementioned process, wherein said separation into a first plurality of fractions comprises washing to remove a liquids fraction, thereby leaving a solids fraction; said liquids fraction comprises mainly hemicellulosic materials in solution; and said hemicellulosic materials are hemicellulose sugars.

In certain embodiments, the invention relates to the aforementioned process, wherein said separation into a first plurality of fractions comprises washing to remove a liquids fraction, thereby leaving a solids fraction; said liquids fraction comprises mainly hemicellulosic materials in solution; said hemicellulosic materials are hemicellulose sugars; and said liquids fraction further comprises residual chemicals applied in the first pretreatment protocol, by-products thereof, degradation products thereof, or combinations thereof.

In certain embodiments, the invention relates to the aforementioned process, wherein said separation into a first plurality of fractions comprises washing to remove a liquids fraction, thereby leaving a solids fraction; said liquids fraction comprises mainly hemicellulosic materials in solution; said hemicellulosic materials are hemicellulose sugars; said liquids fraction further comprises residual chemicals applied in the first pretreatment protocol, by-products thereof, degradation products thereof, or combinations thereof; and said residual chemicals are acid.

In certain embodiments, the invention relates to the aforementioned process, wherein said separation into a first plurality of fractions comprises washing to remove a liquids fraction, thereby leaving a solids fraction; said liquids fraction comprises mainly hemicellulosic materials in solution; said hemicellulosic materials are hemicellulose sugars; said liquids fraction further comprises residual chemicals applied in the first pretreatment protocol, by-products thereof, degradation products thereof, or combinations thereof; said residual chemicals are acid; and said acid is sulfuric acid.

In certain embodiments, the invention relates to the aforementioned process, wherein said separation into a first plurality of fractions comprises washing to remove a liquids fraction, thereby leaving a solids fraction; said liquids fraction comprises mainly hemicellulosic materials in solution; said hemicellulosic materials are hemicellulose sugars;
said liquids fraction further comprises residual chemicals applied in the first pretreatment protocol, by-products thereof, degradation products thereof, or combinations thereof; said residual chemicals are acid; and said degradation products are selected from the group consisting of fermentation inhibitors, acids, furfural, hydroxymethylfurfural, phenols, formic acid, and acetic acid.

In certain embodiments, the invention relates to the aforementioned process, wherein said separation into a first plurality of fractions comprises washing to remove a liquids fraction, thereby leaving a solids fraction; said liquids fraction comprises mainly hemicellulosic materials in solution; said hemicellulosic materials are hemicellulose sugars; and said hemicellulose sugars are glucose, galactose, mannose, xylose, arabinose, or combinations thereof.

In certain embodiments, the invention relates to the aforementioned process, wherein said separation is conducted at about the same temperature as the first pretreatment protocol.

In certain embodiments, the invention relates to the aforementioned process, wherein said second pretreatment protocol comprises heating to a temperature.

In certain embodiments, the invention relates to the aforementioned process, further comprising cooling the heated second product mixture, wherein said second pretreatment protocol comprises heating to a temperature.

In certain embodiments, the invention relates to the aforementioned process, wherein said second pretreatment protocol comprises heating to a temperature; and said temperature is about 160° C. to about 220° C.

In certain embodiments, the invention relates to the aforementioned process, further comprising cooling the heated second product mixture, wherein said second pretreatment protocol comprises heating to a temperature; and said temperature is about 160° C. to about 220° C.

In certain embodiments, the invention relates to the aforementioned process, further comprising cooling the heated second product mixture and processing said second product mixture using enzymatic hydrolysis, wherein said second pretreatment protocol comprises heating to a temperature; and said temperature is about 160° C. to about 220° C.

In certain embodiments, the invention relates to the aforementioned process, further comprising subjecting said second product mixture to biological conversion or chemical conversion.

In certain embodiments, the invention relates to the aforementioned process, further comprising cooling the heated second product mixture, processing said second product mixture using enzymatic hydrolysis, and subjecting said second product mixture to biological conversion or chemical conversion; wherein said second pretreatment protocol comprises heating to a temperature; and said temperature is about 160° C. to about 220° C.

In certain embodiments, the invention relates to the aforementioned process, further comprising cooling the heated second product mixture, processing said second product mixture using enzymatic hydrolysis, and subjecting said second product mixture to biological conversion or chemical conversion; wherein said second pretreatment protocol comprises heating to a temperature; and said temperature is about 160° C. to about 220° C.

In certain embodiments, the invention relates to the aforementioned process, further comprising cooling the heated second product mixture, processing said second product mixture using enzymatic hydrolysis, and subjecting said second product mixture to biological conversion or chemical conversion; wherein said second pretreatment protocol comprises heating to a temperature; said temperature is about 160° C. to about 220° C.; and
said biological conversion comprises enzymatic hydrolysis.

In certain embodiments, the invention relates to the aforementioned process, further comprising cooling the heated second product mixture, processing said second product mixture using enzymatic hydrolysis, and subjecting said second product mixture to biological conversion or chemical conversion; wherein said second pretreatment protocol comprises heating to a temperature; said temperature is about 160° C. to about 220° C.; and said biological conversion is fermentation to afford ethanol.

In certain embodiments, the invention relates to the aforementioned process, further comprising cooling the heated second product mixture, processing said second product mixture using enzymatic hydrolysis, and subjecting said second product mixture to biological conversion or chemical conversion; wherein said second pretreatment protocol comprises heating to a temperature; said temperature is about 160° C. to about 220° C.; and said conversion produces levulinic acid.

In certain embodiments, the invention relates to the aforementioned process, wherein said process is a batch process.

In certain embodiments, the invention relates to the aforementioned process, wherein said process is a continuous process.

In certain embodiments, the invention relates to the aforementioned process, wherein said first pretreatment protocol comprises heating the lignocellulosic materials to a temperature of about 100° C. to about 140° C. in a solution of about 2% to about 5% sulfuric acid for a period of about 30 minutes to about 90 minutes.

In certain embodiments, the invention relates to a process, comprising:
(a) treating lignocellulosic biomass according to a first pretreatment protocol, thereby generating a first product mixture;
(b) separating the first product mixture into a first plurality of fractions; and
(c) treating at least one fraction of said first plurality of fractions according to a second pretreatment protocol, thereby generating a second product mixture; wherein
said lignocellulosic biomass is selected from the group consisting of grass, switch grass, cord grass, rye grass, reed canary grass, miscanthus, sugar-processing residues, sugarcane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, and combinations thereof; and
said first pretreatment protocol comprises heating the lignocellulosic materials to a first temperature in a solution of acid for a first period of time; said separation into a first plurality of fractions comprises washing to remove a liquids fraction, thereby leaving a solids fraction; said second pretreatment protocol comprises heating said solids fraction to a second temperature for a second period of time; said liquids fraction is further processed; and said second product mixture is further processed.

In certain embodiments, the invention relates to the aforementioned process, wherein said first temperature is about 100° C. to about 140° C.

In certain embodiments, the invention relates to the aforementioned process, wherein said first period of time is about 30 minutes to about 90 minutes.

In certain embodiments, the invention relates to the aforementioned process, wherein said solution of acid is of a concentration of about 2% to about 5%.

In certain embodiments, the invention relates to the aforementioned process, wherein said acid is sulfuric acid.

In certain embodiments, the invention relates to the aforementioned process, wherein said solution of acid is of a concentration of about 2% to about 5%; and said acid is sulfuric acid.

In certain embodiments, the invention relates to the aforementioned process, wherein said liquids fraction comprises hemicellulosic materials in solution.

In certain embodiments, the invention relates to the aforementioned process, wherein said liquids fraction comprises hemicellulosic materials in solution; and said hemicellulosic materials are hemicellulose sugars.

In certain embodiments, the invention relates to the aforementioned process, wherein said liquids fraction comprises hemicellulosic materials in solution; said hemicellulosic materials are hemicellulose sugars; and said hemicellulose sugars are glucose, galactose, mannose, xylose, arabinose, or combinations thereof.

In certain embodiments, the invention relates to the aforementioned process, wherein said liquids fraction comprises hemicellulosic materials in solution; and said liquids fraction further comprises residual chemicals applied in the first pretreatment protocol, by-products thereof, degradation products thereof, or combinations thereof.

In certain embodiments, the invention relates to the aforementioned process, wherein said liquids fraction comprises hemicellulosic materials in solution; said liquids fraction further comprises residual chemicals applied in the first pretreatment protocol, by-products thereof, degradation products thereof, or combinations thereof; and said residuals chemicals are acid.

In certain embodiments, the invention relates to the aforementioned process, wherein said liquids fraction comprises hemicellulosic materials in solution; said liquids fraction further comprises residual chemicals applied in the first pretreatment protocol, by-products thereof, degradation products thereof, or combinations thereof; said residuals chemicals are acid; and said acid is sulfuric acid.

In certain embodiments, the invention relates to the aforementioned process, wherein said liquids fraction comprises hemicellulosic materials in solution; said liquids fraction further comprises residual chemicals applied in the first pretreatment protocol, by-products thereof, degradation products thereof, or combinations thereof; and said degradation products are selected from the group consisting of fermentation inhibitors, acids, furfural, hydroxymethylfurfural, phenols, formic acid, and acetic acid.

In certain embodiments, the invention relates to the aforementioned process, wherein said solids fraction comprises cellulosic materials.

In certain embodiments, the invention relates to the aforementioned process, wherein said second temperature is about 160° C. to about 220° C.

In certain embodiments, the invention relates to the aforementioned process, wherein said further processing of the second product mixture comprises enzymatic hydrolysis.

In certain embodiments, the invention relates to the aforementioned process, further comprising subjecting said liquids fraction to biological or chemical conversion.

In certain embodiments, the invention relates to the aforementioned process, further comprising subjecting said liquids fraction to biological or chemical conversion, wherein said biological conversion comprises enzymatic hydrolysis.

In certain embodiments, the invention relates to the aforementioned process, further comprising subjecting said liquids fraction to biological or chemical conversion, wherein said conversion is fermentation to afford ethanol.

In certain embodiments, the invention relates to the aforementioned process, further comprising subjecting said liquids fraction to biological or chemical conversion, wherein said conversion produces levulinic acid.

In certain embodiments, the invention relates to a process, comprising:
(a) treating lignocellulosic biomass according to a first pretreatment protocol, thereby generating a first product mixture;
(b) separating the first product mixture into a first plurality of fractions; and
(c) treating at least one fraction of said first plurality of fractions according to a second pretreatment protocol, thereby generating a second product mixture; wherein
said lignocellulosic biomass is selected from the group consisting of grass, switch grass, cord grass, rye grass, reed canary grass, miscanthus, sugar-processing residues, sugarcane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, and combinations thereof; and
said first pretreatment protocol comprises heating the lignocellulosic materials to a temperature of about 100° C. to about 140° C. in a solution of about 2% to about 5% sulfuric acid for a period of about 30 minutes to about 90 minutes; said separation into a first plurality of fractions comprises washing to remove a liquids fraction, thereby leaving a solids fraction; said second pretreatment protocol comprises heating said solids fraction to a temperature of about 160° C. to about 220° C.; said liquids fraction is further processed; and said second product mixture is further processed.

In certain embodiments, the invention relates to the aforementioned process, wherein said liquids fraction comprises hemicellulosic materials in solution.

In certain embodiments, the invention relates to the aforementioned process, wherein said liquids fraction comprises hemicellulosic materials in solution; and said hemicellulosic materials are hemicellulose sugars.

In certain embodiments, the invention relates to the aforementioned process, wherein said liquids fraction comprises hemicellulosic materials in solution; said hemicellulosic materials are hemicellulose sugars; and said hemicellulose sugars are glucose, galactose, mannose, xylose, arabinose, or combinations thereof

In certain embodiments, the invention relates to the aforementioned process, wherein said liquids fraction comprises hemicellulosic materials in solution; said hemicellulosic materials are hemicellulose sugars; and said liquids fraction further comprises residual chemicals applied in the first pretreatment protocol, by-products thereof, degradation products thereof, or combinations thereof

In certain embodiments, the invention relates to the aforementioned process, wherein said liquids fraction comprises hemicellulosic materials in solution; said hemicellulosic materials are hemicellulose sugars; said liquids fraction further comprises residual chemicals applied in the first pretreatment protocol, by-products thereof, degradation products thereof, or combinations thereof; and said residuals chemicals are acid.

In certain embodiments, the invention relates to the aforementioned process, wherein said liquids fraction comprises hemicellulosic materials in solution; said hemicellulosic materials are hemicellulose sugars; said liquids fraction further comprises residual chemicals applied in the first pretreatment protocol, by-products thereof, degradation products thereof, or combinations thereof; said residuals chemicals are acid; and said acid is sulfuric acid.

In certain embodiments, the invention relates to the aforementioned process, wherein said liquids fraction comprises hemicellulosic materials in solution; said hemicellulosic materials are hemicellulose sugars; said liquids fraction further comprises residual chemicals applied in the first pretreatment protocol, by-products thereof, degradation products thereof, or combinations thereof; and said degradation products are selected from the group consisting of fermentation inhibitors, acids, furfural, hydroxymethylfurfural, phenols, formic acid, and acetic acid.

In certain embodiments, the invention relates to the aforementioned process, wherein said solids fraction comprises cellulosic materials.

In certain embodiments, the invention relates to the aforementioned process, wherein said further processing of the second product mixture comprises enzymatic hydrolysis.

In certain embodiments, the invention relates to the aforementioned process, wherein said further processing of said liquids fraction comprises biological conversion or chemical conversion.

In certain embodiments, the invention relates to the aforementioned process, wherein said further processing of said liquids fraction comprises biological conversion or chemical conversion; and said biological conversion comprises enzymatic hydrolysis.

In certain embodiments, the invention relates to the aforementioned process, wherein said further processing of said liquids fraction comprises biological conversion or chemical conversion; and said conversion is fermentation to afford ethanol.

In certain embodiments, the invention relates to the aforementioned process, wherein said further processing of said liquids fraction comprises biological conversion or chemical conversion; and said conversion produces levulinic acid.

### Exemplification

The invention now being generally described, it will be more readily understood by reference to the following examples which are included merely for purposes of illustration of certain aspects and embodiments of the present invention, and are not intended to limit the invention.

### Prophetic Example 1

### MOD-1 (Modular Ethanol Production Plant)

This module targets high xylan (80%) and low glucan (20%) recovery. It uses low pressure steam (160 PSIG) pre-treatment catalyst, which bypasses solids feeding to high pressure constraint. The mild temperature also minimizes C5 sugar degradation products.

The module uses proven operating conditions and equipment. The operating temperature and pressure are similar to thermo-mechanical pulping. This minimizes technology risk and reduces fixed cost and fast-tracks deployment. Used equipment can be deployed, and no solids feeding development work required.

The module uses high enzyme (xylanase) loading. This minimizes cellulase dependence and reduces operating cost (because cellulase is costly). The short SSCF residence time reduces fixed cost because only smaller vessels sizes are required.

The output solid residue is sold for energy value. For example, the residue can be used as boiler fuel for co-located power producer or as raw material for extrusion to wood pellets.

The architecture is designed for clip-on to optimize performance.

### Prophetic Example 2

### Technology Evolution (Clip on to Modular Ethanol Production Plant)

This module targets high glucan recovery. Here, high pressure steam (250 PSIG) is the pre-treatment catalyst. Pumping solids to high pressure (rather than feeding solids) reduces costs and minimizes degradation products (C5 sugars fermented in MOD-1). Alternatively, this module can use other pre-treatment catalysts like acid or ammonia (injected to zirconium pipe into which solids are pumped). MOD-1 pre-treatment/ethanol concentration may improve recovery of glucan.

This module is deployed by the time cellulase costs have come down. Down-stream CBP organisms will bypass purchased enzyme constraint.

### INCORPORATION BY REFERENCE

All of the U.S. patents and U.S. published patent applications cited herein are hereby incorporated by reference. U.S. Pat. Nos. 5,916,787, 5,482,846, 6,333,181, and 5,000,000 are hereby incorporated by reference. In addition, U.S. Pat. No. 4,600,590 is hereby incorporated by reference; U.S. Pat. No. 5,037,663 is hereby incorporated by reference; U.S. Pat. No. 5,171,592 is hereby incorporated by reference; U.S. Pat. No. 4,356,196 is hereby incorporated by reference; and U.S. Pat. No. 5,865,898 is hereby incorporated by reference. U.S. Pat. Nos. 4,897,947, 4,236,021, 3,663,368, 5,859,263, 5,608,105, 7,153,996 and 6,054,611 are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A process, comprising:
(a) treating lignocellulosic biomass according to a first pretreatment protocol, thereby generating a first product mixture;
(b) separating the first product mixture into a first plurality of fractions; and
(c) treating at least one fraction of said first plurality of fractions according to a second pretreatment protocol, thereby generating a second product mixture.

2-6. (canceled)

7. The process of claim 1,
wherein
said lignocellulosic biomass is selected from the group consisting of grass, switch grass, cord grass, rye grass, reed canary grass, miscanthus, sugar-processing residues, sugarcane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, and combinations thereof

8. The process of claim 7, wherein said first pretreatment protocol or second pretreatment protocol comprises ball-milling, two-roll milling, hammer milling, colloid milling, high pressure, steaming, high energy, radiation, pyrolysis, sodium hydroxide, calcium hydroxide, ammonia, sulfuric acid, hydrochloric acid, hydrofluoric acid, chlorine dioxide, nitrogen dioxide, sulfur dioxide, hydrogen peroxide, ozone, cellulose solvents, ethanol-water extraction, benzene-ethanol extraction, steam explosion, AFEX, recombinant microorganisms, or a combination thereof.

9. The process of claim 7, wherein said first pretreatment protocol comprises heating the lignocellulosic materials to a temperature in a solution of acid for a period of time.

10. The process of claim 7, wherein
said first pretreatment protocol comprises heating the lignocellulosic materials to a temperature in a solution of acid for a period of time; and
said acid is sulfuric acid.

11. The process of claim 7, wherein
said first pretreatment protocol comprises heating the lignocellulosic materials to a temperature in a solution of acid for a period of time; and
said temperature is about 100° C. to about 140° C.

12. The process of claim 7, wherein
said first pretreatment protocol comprises heating the lignocellulosic materials to a temperature in a solution of acid for a period of time; and
said period of time is about 30 minutes to about 90 minutes.

13. The process of claim 7, wherein
said first pretreatment protocol comprises heating the lignocellulosic materials to a temperature in a solution of acid for a period of time; and
said solution of acid is of a concentration of about 2% to about 5%.

14-15. (canceled)

16. The process of claim 7, wherein said separation into a first plurality of fractions comprises washing to remove a liquids fraction, thereby leaving a solids fraction.

17. The process of claim 7,
wherein
said separation into a first plurality of fractions comprises washing to remove a liquids fraction, thereby leaving a solids fraction; and
said solids fraction comprises cellulosic materials.

18. The process of claim 7, wherein
said separation into a first plurality of fractions comprises washing to remove a liquids fraction, thereby leaving a solids fraction; and
said liquids fraction comprises mainly hemicellulosic materials in solution.

19. The process of claim 7, wherein
said separation into a first plurality of fractions comprises washing to remove a liquids fraction, thereby leaving a solids fraction;
said liquids fraction comprises mainly hemicellulosic materials in solution; and
said hemicellulosic materials are hemicellulose sugars.

20. The process of claim 7, wherein
said separation into a first plurality of fractions comprises washing to remove a liquids fraction, thereby leaving a solids fraction;
said liquids fraction comprises mainly hemicellulosic materials in solution;
said hemicellulosic materials are hemicellulose sugars; and
said liquids fraction further comprises residual chemicals applied in the first pretreatment protocol, by-products thereof, degradation products thereof, or combinations thereof.

21-25. (canceled)

26. The process of claim 7, wherein said second pretreatment protocol comprises heating to a temperature, thereby forming a heated second product mixture.

27. The process of claim 26, further comprising cooling the heated second product mixture, wherein said second pretreatment protocol comprises heating to a temperature.

28. The process of claim 26, wherein
said second pretreatment protocol comprises heating to a temperature; and
said temperature is about 160° C. to about 220° C.

29. (canceled)

30. The process of claim 26, further comprising cooling the heated second product mixture and processing said second product mixture using enzymatic hydrolysis, wherein
said second pretreatment protocol comprises heating to a temperature; and
said temperature is about 160° C. to about 220° C.

31. The process of claim 26, further comprising subjecting said second product mixture to biological conversion or chemical conversion.

32. The process of claim 26, further comprising cooling the heated second product mixture, processing said second product mixture using enzymatic hydrolysis, and subjecting said second product mixture to biological conversion or chemical conversion; wherein
said second pretreatment protocol comprises heating to a temperature; and
said temperature is about 160° C. to about 220° C.

33-38. (canceled)

39. The process of claim 7, wherein
said first pretreatment protocol comprises heating the lignocellulosic materials to a first temperature in a solution of acid for a first period of time; said separation into a first plurality of fractions comprises washing to remove a liquids fraction, thereby leaving a solids fraction; said second pretreatment protocol comprises heating said solids fraction to a second temperature for a second period of time; said liquids fraction is further processed; and said second product mixture is further processed.

40-58. (canceled)

59. The process of claim 7, wherein
said first pretreatment protocol comprises heating the lignocellulosic materials to a temperature of about 100° C. to about 140° C. in a solution of about 2% to about 5% sulfuric acid for a period of about 30 minutes to about 90 minutes; said separation into a first plurality of fractions comprises washing to remove a liquids fraction, thereby leaving a solids fraction; said second pretreatment protocol comprises heating said solids fraction to a temperature of about 160° C. to about 220° C.; said liquids fraction is further processed; and said second product mixture is further processed.

60-72. (canceled)
